(19) United States
(12) Patent Application Publication
(10) Pub. No.: US 2021/0322042 A1
(43) Pub. Date: Oct. 21, 2021

(54) SURGICAL INSTRUMENTS WITH REDUCED CAPACITANCE, RELATED DEVICES, AND RELATED METHODS

(71) Applicant: INTUITIVE SURGICAL OPERATIONS, INC., Sunnyvale, CA (US)

(72) Inventors: Jordan M. KLEIN, II, Palo Alto, CA (US); Thomas G. COOPER, Menlo Park, CA (US); Samuel T. CREWS, Palomar Park, CA (US); Cara C.H. MOTOOKA, San Jose, CA (US)

(73) Assignee: INTUITIVE SURGICAL OPERATIONS, INC., Sunnyvale, CA (US)

(21) Appl. No.: 17/260,597

(22) PCT Filed: Jul. 12, 2019
(86) PCT No.: PCT/US2019/041595

§ 371 Date: Jan. 15, 2021

Related U.S. Application Data
(60) Provisional application No. 62/699,193, filed on Jul. 17, 2018.

(51) Int. Cl. A61B 17/28

(52) U.S. Cl. CPC A61B 17/282; A61B 2017/00929

(57) ABSTRACT

An instrument comprises a plurality of elongate members having a proximal end and a distal end, a shaft, an actuation member extending through the shaft, and a tube member extending through the shaft and housing at least a portion of a length of the actuation members. An end effector is coupled to a distal end of, and a force transmission mechanism is coupled to a proximal region of, at least one of the plurality of elongate members. At least one of the plurality of elongate members comprises a first electrically conductive length portion, a second electrically conductive length portion, and an electrically insulative length portion between and connecting the first electrically conductive length portion and the second electrically conductive length portion. The electrically insulative length portion reduces a conductive length of the elongate member, thereby reducing a capacitive coupling effect in the instrument.

### CROSS-REFERENCE TO RELATED APPLICATIONS

This application claims priority to U.S. Provisional Application No. 62/699,193, filed Jul. 17, 2018, which is incorporated by reference herein in its entirety.

### TECHNICAL FIELD

Aspects of the present disclosure relate to instruments that are remotely actuatable via actuation members that transmit actuation forces from a force drive transmission at one end of a shaft of the instrument to a moveable end effector or other component at the other end of the instrument shaft. In particular, aspects of the present disclosure relate to surgical instruments, and related methods and systems.

### INTRODUCTION

Various surgical instruments can be used in an operating site to carry out a surgical procedure. Surgical instruments may be energized (e.g. to perform electrosurgical procedures through the application of an electrical current), or may be non-energized (e.g. to grip or cut tissue using mechanical actuation). Such surgical instruments may include, without limitation, minimally invasive surgical instruments configured for manual, laparoscopic use or as part of a teleoperated surgical system. One example of a teleoperated, computer-assisted surgical system (e.g., a robotic system that provides telepresence), is the da Vinci® Surgical System manufactured by Intuitive Surgical, Inc. of Sunnyvale, Calif.

In some cases, multiple surgical instruments are in use at the surgical site. Such surgical instruments, whether energized (i.e. “hot”) or non-energized (i.e. “cold”), may include electrically conductive components, including for example, components made of conductive materials, such as, for example, metals or metal alloys. Many of these surgical instruments also include actuation members, such as cables, rods, etc., or combinations thereof configured to transmit tensile and/or compressive forces from a force transmission device operably coupled at a proximal region of a surgical instrument shaft to an actuatable component, such as an end effector or articulating wrist mechanism, operably coupled at a distal region of the surgical instrument shaft. Such actuatable components may also be electrically conductive and/or made of conductive materials, such as, metals or metal alloys. If an energized or “hot” electrosurgical instrument is close to, or touching, a conductive, non-energized or “cold” instrument using such components, there exists a potential to transmit electrical energy from the energized instrument to the non-energized instrument. For example, a “hot” instrument may accidentally or intentionally make contact with a “cold” instrument, resulting in electrical energy being conducted by conductive components of the “cold” instrument. Further, a “hot” instrument being used in close proximity to a “cold” instrument may induce electrical energy in the “cold” instrument. As a consequence of energizing a component of the cold instrument, additional components of the cold instrument may become energized, for example through capacitive coupling. For example, if an actuating rod or cable of the “cold” instrument is energized by a “hot” instrument accidentally or intentionally making contact with an end-effector of the “cold” instrument, the now-energized actuating rod or cable can induce electrical energy through capacitive coupling in other conductive portions of the instrument, for example, including a shaft, wrist structure, or other exposed portion of the instrument.

Further, even other energized instruments can be susceptible to such undesired electrical effects from an energized instrument. For example, a monopolar instrument uses voltages significantly higher than a bipolar instrument. A bipolar instrument in close proximity to a monopolar instrument may be susceptible to electrical energy from the monopolar instrument, causing undesired capacitive coupling within conductive portions of the bipolar instrument. For the purposes of this disclosure, a “cold” instrument hereinafter refers to any instrument that becomes energized, whether intentionally or unintentionally, by another energized instrument as opposed to being directly energized from an energy source.

While insulative sheaths can offer some level of protection against electrical energy being conducted from internal components to exposed electrically conductive components, such as the instrument shaft and wrist structures, it may be desirable to further mitigate or prevent the potential for electrical capacitive coupling between electrically conductive components within the instrument and exposed electrically conductive components of the instrument.

A need exists to provide electrical insulation in non-energized instruments (i.e., instruments that are not electrosurgical instruments) so as to prevent or mitigate capacitive coupling between portions of such an instrument that may be temporarily subjected to electrical energy due to its proximity to energized instruments. A need also exists to prevent or mitigate a capacitive coupling between portions of a surgical instrument without compromising the durability or reliability of the instrument. A need exists to continue to use relatively strong metal or metal alloy materials, for example, for actuation members, while preventing or mitigating undesirable electrically conductive pathways in a surgical instrument.

### SUMMARY

Exemplary embodiments of the present disclosure may solve one or more of the above-mentioned problems and/or may demonstrate one or more of the above-mentioned desirable features. Other features and/or advantages may become apparent from the description that follows.

In accordance with at least one exemplary embodiment, an actuation member for transmitting force from a drive mechanism along a shaft of an instrument includes a first electrically conductive length portion, a second electrically conductive length portion, and an electrically insulative length portion disposed between and connecting the first electrically conductive length portion and the second electrically conductive length portion.

In accordance with at least another exemplary embodiment, an instrument includes a shaft having a proximal end and a distal end, an end effector coupled to and extending in a direction distally away from the distal end of the shaft, a force transmission mechanism coupled to a proximal region of the shaft, and an actuation member extending through the shaft and being operably coupled to the force transmission mechanism at one end and to a moveable component of the instrument at an opposite end. The actuation member includes a first electrically conductive length portion, a second electrically conductive length portion, and an electrically insulative length portion disposed between and connecting the first electrically conductive length portion and the second electrically conductive length portion.

In accordance with yet another exemplary embodiment, a method of reducing a conductive length of an actuation member for transmitting an actuation force from a drive mechanism to an end effector of a surgical instrument includes forming a first proximal portion of the actuating member with an electrically conductive material, wherein a proximal end of the first proximal portion is configured to be operably coupled to the drive mechanism, forming a first distal portion of the actuating member with the electrically conductive material, wherein a distal end of the first distal portion is configured to be operably coupled to the end effector, and providing a first electrically insulative material in between the first proximal portion and the first distal portion. The first electrically insulative material electrically insulates the first proximal portion from electrical energy in the first distal portion.

In accordance with yet another exemplary embodiment, an instrument includes a plurality of elongate members having a proximal end and a distal end, the plurality of elongate members comprising at least a shaft, an actuation member extending through the shaft, and a tube member extending through the shaft and housing at least a portion of a length of the actuation members, an end effector coupled to a distal end of at least one of the plurality of elongate members, and a force transmission mechanism coupled to a proximal region of at least one of the plurality of elongate members, wherein at least one of the plurality of elongate members includes a first electrically conductive length portion, a second electrically conductive length portion, and an electrically insulative length portion disposed between and connecting the first electrically conductive length portion and the second electrically conductive length portion.

Additional objects, features, and/or advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are exemplary and explanatory only and are not restrictive of the claims; rather the claims should be entitled to their full breadth of scope, including equivalents.

### BRIEF DESCRIPTION OF THE DRAWINGS

The present disclosure can be understood from the following detailed description, either alone or together with the accompanying drawings. The drawings are included to provide a further understanding of the present disclosure, and are incorporated in and constitute a part of this specification. The drawings illustrate one or more exemplary embodiments of the present teachings and together with the description serve to explain certain principles and operation.

FIG. 1 is a perspective view of an exemplary embodiment of a surgical instrument.

FIG. 2 is a cross-sectional view of an exemplary embodiment of a surgical instrument comprising an actuation member having an electrically insulative portion in accordance with the present disclosure.

FIG. 3 is a partial, detailed cross-sectional view of an exemplary embodiment of a surgical instrument comprising an actuation member having an electrically insulative portion in accordance with the present disclosure.

FIG. 4 is a cutaway schematic view of an exemplary embodiment of an actuation member having an electrically insulative portion in accordance with the present disclosure.

FIG. 5 is a partial, detailed cross-sectional view of another exemplary embodiment of a surgical instrument comprising an actuation member having an electrically insulative portion in accordance with the present disclosure.

FIGS. 6A and 6B are cutaway schematic views of an exemplary embodiment of an actuation member having an electrically insulative portion in accordance with the present disclosure.

FIG. 7 is a perspective view of an exemplary embodiment of a surgical instrument comprising a plurality of jointed portions.

FIG. 8 is a perspective, diagrammatic view of an exemplary embodiment of a surgical instrument comprising a shaft having an electrically insulative portion in accordance with the present disclosure.

### DETAILED DESCRIPTION

The present disclosure contemplates various exemplary embodiments of surgical instruments and related devices configured for electrical isolation between portions of a component of a surgical instrument that are made of an electrically conductive material. For example, according to exemplary embodiments of the disclosure, a surgical instrument may include an actuation member comprising an electrically conductive material, (e.g., a metal and/or metal alloy) with an electrically insulative material disposed to provide an insulative “break” in a conductive pathway between the electrically conductive material portions of the instrument. In some exemplary embodiments, the actuation member includes at least two electrically conductive portions and an electrically insulative portion disposed in between the two electrically conductive portions. In other embodiments, the electrically insulative portion may be disposed anywhere along a length of one or more electrically conductive portions between a distal end of the one or more electrically conductive portions and a proximal end of the one or more electrically conductive portions.

The exemplary embodiments disclosed herein thus provide actuation members that can achieve electrical isolation between a distal end and a proximal end of the actuation members. Moreover, actuation members in accordance with various exemplary embodiments of the present disclosure have a relatively short conductive pathway length from, for example, one end of the actuation member to a portion along the length of the actuation member where the electrically insulative “break” occurs. Because the amount of capacitive coupling that may be induced in other electrically conductive components of the surgical instrument, such as the instrument shaft, including any joint mechanisms, is proportional to a conductive length of the actuation member, the shorter conductive pathway of these exemplary actuation members reduces or mitigates a capacitive coupling effect. Such shorter conductive pathways minimize (or eliminate) unintended conduction of electrical energy between the actuation member and other conductive components of the surgical instrument or regions external to the instrument.

In various exemplary embodiments, an electrically insulative “break” may be provided in components of an instrument other than the actuation member. For example, an instrument shaft may have one or more “breaks” provided at different portions along a length of the instrument shaft. Since the amount of capacitive coupling is further proportional to the length of the instrument shaft itself, reducing a conductive length of the shaft into two or more conductive portions (that are electrically insulated from each other) can further reduce a capacitive coupling effect induced within the instrument shaft by other energized components of the instrument such as, for example, an energized actuation member.

In addition, electrically isolating different portions of such surgical instruments may be difficult for various reasons. For example, surgical instruments such as clamps, forceps, grippers, shears, etc. are often configured to deliver relatively high magnitudes of force to carry out desired surgical operations. The actuation members must be able to transmit such actuating forces from a force transmission mechanism to an end effector or other moveable component of the surgical instrument along an entire length of the actuation member. To withstand such forces and provide durability, the actuation members of such surgical tools may be constructed from metals or metal alloys such as stainless steel, titanium alloys, aluminum alloys, etc., based on material properties such as yield strength, toughness, hardness, etc. Such materials, however, are typically relatively highly electrically conductive, which increases the aforementioned electrical capacitive coupling effect. Thus, various exemplary embodiments described herein provide electrically insulative materials disposed along a length of the actuation members that can maintain durability of the actuation member while retaining a relatively small outer dimension on the order of dimensions of a single electrically conductive material actuation member (such as a metal or metal alloy actuation member). In other words, exemplary embodiments described herein permit the force transmission (compressive and tensile strength) in an actuation member that provides a level of electrical insulation between proximal and distal portions of the actuation member.

Exemplary embodiments described herein may be used, for example, with teleoperated, computer-assisted surgical systems (sometimes referred to as robotic surgical systems) such as those described in, for example, U.S. Patent App. Pub. No. US 2013/0325033 A1, entitled “Multi-Port Surgical Robotic System Architecture” and published on Dec. 5, 2013, U.S. Patent App. Pub. No. US 2013/0325031 A1, entitled “Redundant Axis and Degree of Freedom for Hardware-Constrained Remote Center Robotic Manipulator” and published on Dec. 5, 2013, and U.S. Pat. No. 8,852,208, entitled “Surgical System Instrument Mounting” and published on Oct. 7, 2014, each of which is hereby incorporated by reference in its entirety. Further, the exemplary embodiments described herein may be used, for example, with a da Vinci® Surgical System, such as the da Vinci Si® Surgical System or the da Vinci Xi® Surgical System, both with or without Single-Site® single orifice surgery technology, all commercialized by Intuitive Surgical, Inc. Although various exemplary embodiments described herein are discussed with regard to surgical instruments used with a patient side cart of a teleoperated surgical system, the present disclosure is not limited to use with surgical instruments for a teleoperated surgical system. For example, various exemplary embodiments of actuation members described herein can optionally be used in conjunction with other laparoscopic surgical instruments, including hand-held, manual surgical instruments, or with other surgical applications.

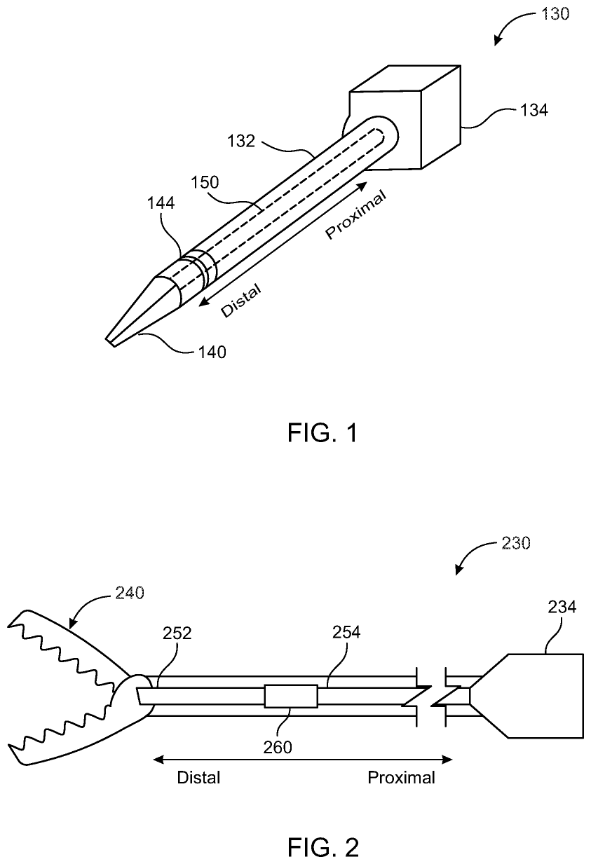

FIG. 1 shows a perspective schematic diagram of an exemplary surgical instrument 130. The surgical instrument 130 comprises a shaft 132 with an end effector 140 positioned at a distal end region thereof. (The distal and proximal directions as used herein are defined relative to the instrument as shown by the labeling in FIG. 1). In an exemplary embodiment, the end effector 140 includes jaws configured to perform, e.g., a gripping function. However, those having ordinary skill in the art would appreciate that other end effector configurations are contemplated, such as those used as forceps, a grasper, a needle driver, a scalpel, scissors, a stapler, a clamp, a cauterizing tool, a hook, a blade, etc. The shaft 132 may optionally include a wrist portion 144 that enables articulation of end effector 140 in one or more directions. For example, a force transmission mechanism 134 may generate an actuating force that is transmitted via an actuation member 150 to actuate or articulate wrist portion 144, end effector 140, or other portions of surgical instrument 130, as further described herein. The diameter or diameters of shaft 132, one or more optional wrist mechanisms 144, and end effector 140 are generally selected according to the size of the cannula or other guide structure with which surgical instrument 130 is intended to be used, and depending on the surgical procedures being performed. In various exemplary embodiments, a shaft 132 and/or wrist mechanism 144 has a diameter of ranging from about 4 mm to about 10 mm, for example, about 5 mm to about 8 mm.

The actuation member 150 may be positioned within a central bore of shaft 132. The actuation member 150 is configured to transmit an actuating force generated at force transmission mechanism 134. For example, actuation member 150 may comprise a compression rod-like member or cable member capable of transmitting tensile (i.e. pulling) and/or compressive (i.e. pushing) forces to actuate other components of surgical instrument 130, such as end-effector 140, or wrist portion 144. Further, the actuation member 150 may include a first component at a proximal end of the actuation member 150 configured to interact with the force transmission mechanism 134, for example, with a drive mechanism of the force transmission mechanism 134. The drive mechanism delivers an actuation force that is transmitted along the actuation member 150 to end effector or other moveable component, such as the wrist portion 144, provided at a distal end region of the surgical instrument 130. Thus, the actuation member 150 may further include a second component towards a distal end of the actuation member 150 configured to interact with, for example, the end effector 140, the wrist portion 144, etc.

Further, as described herein, the actuation member may comprise an electrically conductive material, (e.g., a metal and/or metal alloy) with an electrically insulative material disposed along a portion of the length of the actuation member so as to form an electrically insulative break in a conductive pathway between the electrically conductive material portions of the actuation member. The portion of the actuation member comprising the electrically insulative material (hereinafter referred to as the electrically insulative portion) is configured to transmit the actuating force along the entire length of the actuation member from a proximal portion of the actuation member to a distal portion of the actuation member. In some exemplary embodiments, the actuation member includes a proximal electrically conductive portion and a distal electrically conductive portion, with the electrically insulative portion disposed in between these electrically conductive portions of the actuation member. For example, the electrically insulative portion may be disposed anywhere along a length of the actuation member, and may further be disposed within a length of one or both of the proximal electrically conductive portion and the distal electrically conductive portion.

FIG. 2 is a cross-sectional view of an exemplary embodiment of a surgical instrument 230 including an actuation member 250 that comprises an electrically insulative portion 260 along a length of the actuation member 250. Electrically insulative portion 260 provides an electrical “break” or isolation between a distal portion and a proximal portion of the actuation member 250. For example, actuation member 250 comprises an electrically conductive material, (e.g., a metal and/or metal alloy), and electrically insulative portion 260 may comprise a non-conductive material disposed within at least a portion of the metal and/or metal alloy, thereby forming a break in a conductive pathway between proximal and distal portions of the actuation member 250. Further, electrically insulative portion 260 is formed from a nonconductive material that is sufficiently strong to transmit an actuating force (including both compressive and tensile forces) that are delivered from drive mechanism 234 along the actuation member to an end effector 240. Suitable materials for electrically insulative portion 260 may include, but are not limited to, for example, a thermoplastic such as Amodel. Amodel is useful for numerous reasons including, but not limited to, its tensile strength and dielectric strength. For example, a minimum insulative thickness of a material is dependent on a material dielectric strength. Amodel has a dielectric strength of approximately 500 Volts/0.001 in. (mil)-800 Volts/0.001 in. (mil). A monopolar surgical instrument may be energized at between about 1000 Volts-3000 Volts. Thus, a minimum thickness of Amodel to achieve a dielectric strength for 3000 Volts is approximately 0.006 in. of Amodel. In other exemplary embodiments, materials that may be used as an electrically insulative break include high performance polyaryletherketones such as PEEK, lay-up composite polymers such as KyronMAX, epoxy-fiberglass combinations, and ceramics such as Alumina.

Placement of the electrically insulative portion 260 can thus serve to reduce a length of a conductive pathway along the actuation member between the end effector 240 (or other actuatable component) and the force transmission mechanism at the proximal region of surgical instrument 230. In turn, an amount of capacitive coupling from an actuation member to the exposed electrically conductive materials of the instrument can be reduced or prevented because such capacitive coupling is proportional to a conductive length of the actuation member. That is, the shorter conductive pathway resulting from the inclusion of the electrically insulative “break” in the actuation member reduces or mitigates a capacitive coupling effect and strength.

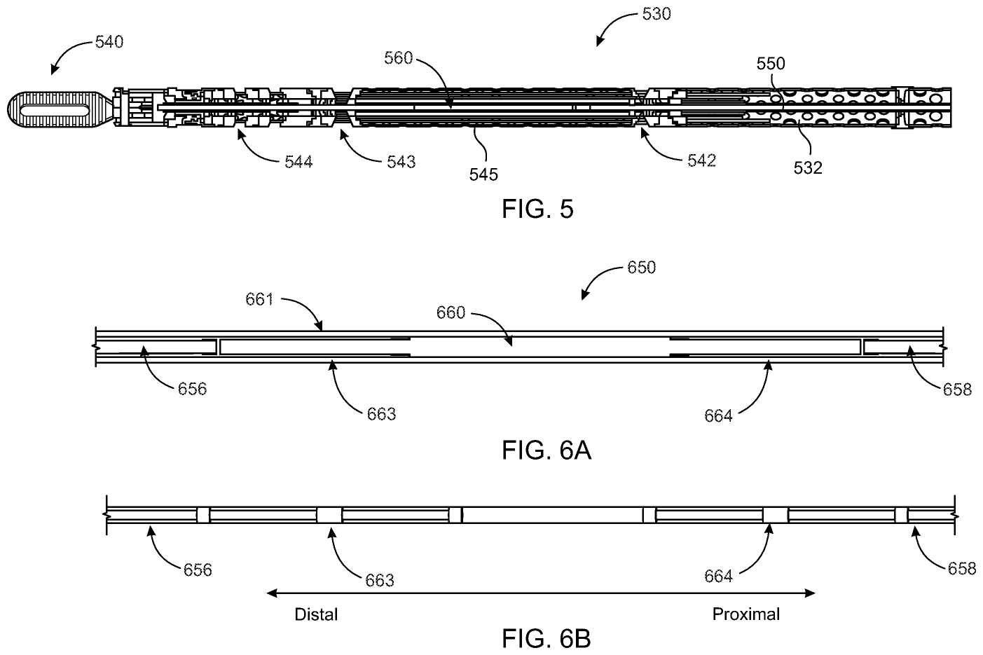

In an exemplary embodiment, creating an electrical break approximately 6 inches from the location at which the actuation member engages an end effector, for example, can reduce the overall capacitance of the instrument from approximately 100 pF to less than 15 pF. In other exemplary embodiments, the electrical break may be positioned distally to within approximately 3 inches of the distal end, where space is significantly more constrained (as further described in, for example, FIGS. 5-7). Thus, the non-conductive material for electrically insulative portions in such embodiments exhibits sufficient strength without having to significantly increase the outer diameter of the actuation member along the length portion the electrically insulative material is used. Further, the more distal location of the insulative portion may reduce the overall capacitance from approximately 100 pF to less than 5 pF. Generally, the capacitance induced within the overall instrument reduces linearly with the proximity of the insulative portion to the distal end of the instrument.

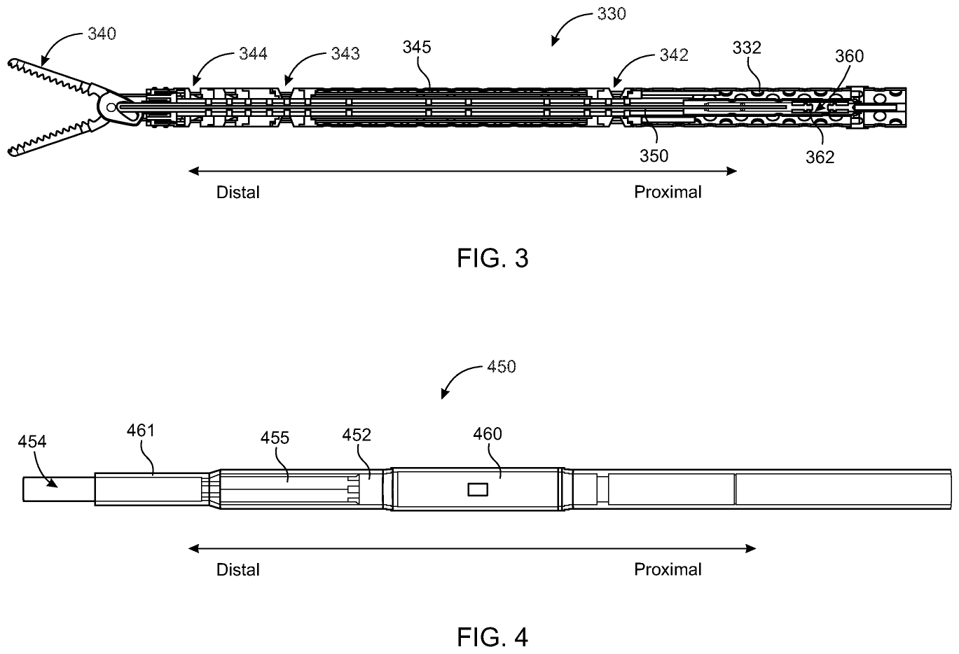

FIG. 3 is a detailed cross-sectional view of an exemplary embodiment of a surgical instrument 330 including an actuation member 350 comprising an electrically insulative portion 360 for providing an insulative “break” in a conductive pathway of the actuation member 350. The surgical instrument 330 includes a shaft 332 with an end effector 340 positioned at a distal end thereof. In an exemplary embodiment, the end effector 340 includes jaws configured to perform, e.g., a gripping function. However, those having ordinary skill in the art would appreciate that other end effector configurations are contemplated, such as those used as forceps, a grasper, a needle driver, a scalpel, scissors, a stapler, a clamp, a cauterizing tool, a hook, a blade, etc. The surgical instrument 330 also includes an actuation member 350 that positioned within a central bore of shaft 332, and is configured to translate distally and proximally relative to shaft 332, and to transmit actuating forces from a force transmission mechanism (not shown herein) to other components of surgical instrument 330 such as, for instance, end effector 340. Further, shaft 332 may include one or more joint structures that impart one or more degrees of freedom to the end effector 340. Such a combination of joint structures may be referred to as a parallel motion linkage mechanism. For example, the shaft 332 includes (in proximal-to-distal order) a first pitch and/or yaw joint 342, a joint tube portion 345, and a second pitch and/or yaw joint 343. Although not shown herein, additional cables or actuation members may extend through the shaft 332 to connect the first and second pitch/yaw joints, which are disposed at the opposite ends of the joint tube portion 345. The additional cables are used to actuate the pitch/yaw joints 342, 343 in combination with tube portion 345 to move end effector 340 laterally with reference to a longitudinal axis of shaft 332, without changing an orientation of end effector 340. Further, wrist portion 344 (located distally from said parallel motion linkage mechanism) can be used to change an orientation of end effector 340 in various degrees of freedom (DOF).

Thus, actuation member 350 may be configured to transmit the actuating forces to actuate or articulate one or more of joint structures 342, 343, and 344. In other exemplary embodiments, a plurality of actuation members may be provided within shaft 332, and configured to actuate one or more components of surgical instrument 330, including end effectors 340 and joint structures 342, 343, 344. Further, a proximal portion of an actuation member 350 extending through a non-jointed portion of shaft 332 may be relatively rigid to be able to interface with and transmit actuation forces, while a distal portion of the actuation member 350 extending through joint structures 342, 343, and/or 344 may comprise a flexible portion. The flexible portion of the actuation member 350 may comprise a cable (e.g., such as twisted or braided strands of metal or metal alloy), having a degree of flexibility sufficient to enable the flexible portion to flex (e.g., bend) with the translation and/or articulation of joint structures 342, 343, 344.

Further, as described herein, the actuation member 350 may comprise an electrically conductive material (e.g., a metal and/or metal alloy), and an electrically insulative material 360 disposed in between electrically conductive portions, thereby forming an insulative break in an electrically conductive pathway between proximal and distal portions of the actuation member 350. The portion of the actuation member 350 comprising the electrically insulative material (i.e., the electrically insulative portion 360) is made of a material that is electrically insulative, while being strong enough to transmit the actuating forces (i.e. tensile and/or compressive forces) between the electrically conductive portions of the actuation member 350. As described above, the electrically insulative portion 360 may be disposed anywhere along a length of the actuation member 350. In this exemplary embodiment, the electrically insulative portion 360 is disposed proximally relative to the pitch/yaw joint 342. For example, the electrically insulative portion 360 includes overmolds 362 on either side of electrically insulative portion 360, that enable electrically insulative portion 360 to be securely coupled to actuation member 350, thereby being able to transmit actuation forces along the proximal-distal direction. Suitable materials for electrically insulative portion 360 and overmolds 362 may include, but are not limited to, for example, a thermoplastic such as Amodel.

FIG. 4 is a schematic view of an exemplary embodiment of an electrically conductive proximal portion of an actuation member 450 comprising a rigid proximal portion 452, a flexible distal portion 454, and an electrically insulative portion 460 that provides electrical isolation or a “break” between distal and proximal ends of actuation member 450. As described above (for example, with respect to FIG. 3), a distal portion of an end effector may be flexible in order to transmit actuating forces through jointed portions of a shaft, while a proximal portion of the end effector may be rigid. In this embodiment, distal portion 454 and proximal portion 452 are physically coupled using a crimp 455. Portions 452 and 454 of actuation member 450 may comprise an electrically conductive material, (e.g., a metal and/or metal alloy), and electrically insulative portion 460 may comprise a non-conductive material, thereby forming an electrical break in a conductive pathway between distal and proximal ends of actuation member 450. Further, electrically insulative portion 460 is made of a nonconductive material that is sufficiently strong to transmit an actuating force (including compressive and tensile forces) that are delivered from a drive mechanism to other components of a surgical instrument, such as an end effector or a wrist (or other joint structure).

Thus, a length of a conductive pathway along actuation member 450 is reduced by provision of electrically insulative portion 460. As an amount of capacitive coupling induced in the actuation member 450 is proportional to a conductive length of the actuation member 450, the shorter conductive pathway of the disclosed embodiment minimizes a capacitive coupling effect in a direction proximal from insulative portion 460. Further, an electrically insulative sleeve 461 is disposed over a portion of actuation member 450, extending distally beyond crimp 455 so as to cover electrically insulative portion 460, thereby forming a continuous electrically insulated outer surface. The electrically insulative sleeve 461 comprises a sleeve (e.g., tube) of material configured to tightly contract around actuation member 450. For example, the electrically insulative material may be heat-shrink tubing made of, for example, nylon, polyolefin, or other heat-shrinkable and electrically insulative polymer materials.

In the embodiments described above (for example, with reference to FIGS. 3-4), there is ample space or tolerance for coupling using overmolds (such as, for example, overmolds 362). However, an amount of space or tolerance may be reduced towards the distal end of a shaft, particularly within (and in between) joint portions and wrists. For example, in an articulating section of the instrument (i.e. within the jointed portions of a shaft), more components (e.g., actuation members) may be needed for actuation of the various joints, which reduces a cross-sectional area towards the distal portion of the shaft. Thus, the exemplary embodiments of FIGS. 5-7 described below illustrate alternative materials for exemplary insulative portions and methods for coupling thereof.

FIG. 5 is a detailed cross-sectional view of an exemplary embodiment of a surgical instrument 530 including an actuation member 550 that comprises an electrically insulative portion 560. The surgical instrument 530 includes a shaft 532 with an end effector 540 positioned at a distal end thereof. In an exemplary embodiment, the end effector 540 includes jaws configured to perform, e.g., a gripping function. However, those having ordinary skill in the art would appreciate that other end effector configurations are contemplated, such as those used as forceps, a grasper, a needle driver, a scalpel, scissors, a stapler, a clamp, a cauterizing tool, a hook, a blade, etc. The surgical instrument 530 also includes an actuation member 550 that positioned within a central bore of shaft 532, and is configured to translate distally and proximally relative to shaft 532, and to transmit actuating forces from a force transmission mechanism (not shown herein) to other components of surgical instrument 530 such as, for instance, end effector 540. A distance of travel of the actuation member 550 in the proximal-distal direction, in order to actuate said components, may hereinafter be referred to as a “throw” of end effector 540 (and portions thereof).

Further, shaft 532 may include one or more joint structures that impart one or more degrees of freedom to the end effector 540. For example, shaft 532 includes a pitch/yaw joints 542 and 543, and a wrist portion 544. Thus, actuation member 550 may be configured to transmit the actuating forces to actuate or articulate one or more of joint structures 542, 543, and 544, so as to enable translation and articulation of end effector 540 in various directions or degrees of freedom (DOF). In other exemplary embodiments, a plurality of actuation members may be provided within shaft 532, and configured to actuate one or more components of surgical instrument 530, including end effectors 540 and joint structures 542, 543, 544. Further, a proximal portion of an actuation member 550 extending through a non-jointed portion of shaft 532 may be relatively rigid to be able to interface with and transmit actuation forces, while a distal portion of the actuation member 550 extending through joint structures 542, 543, and/or 544 may comprise a flexible portion. The flexible portion of the actuation member 550 may comprise a cable (e.g., such as twisted or braided strands of metal or metal alloy), having a degree of flexibility sufficient to enable the flexible portion to flex (e.g., bend) with the translation and/or articulation of joint structures 542, 543, 544 and end effector 540.

Further, as described herein, the actuation member 550 may comprise an electrically conductive material (e.g., a metal and/or metal alloy), and an electrically insulative material 560 disposed in between electrically conductive portions, thereby forming an insulative break in an electrically conductive pathway between proximal and distal portions of the actuation member 550. As described above, the electrically insulative portion 560 may be disposed anywhere along a length of the actuation member 550. In this exemplary embodiment, the electrically insulative portion 560 is disposed distally relative to the pitch/yaw joint 542, i.e. within a portion of actuation member 550 that is housed within joint tube 545. Further, electrically insulative portion 560 is a rigid non-conductive material, so as to enable transmission of force between flexible metallic portions 556, 558. As further described herein, despite flexible portions 556, 558 of the actuation member being routed through the aforementioned joint structures, electrically insulative portion 560 may be disposed within a portion of the length of the actuation member that remains straight during a range of motion (or “throw”) of the actuation member. For example, the electrically insulative portion 560 may be disposed in a region corresponding to a joint tube portion 545. Generally, a “throw” of the actuation member can be between 0.1 in. to 0.5 in. depending on instrument type. Exemplary “cold” instruments incorporating the described actuation members may have a “throw” of approximately 0.125 in.+/−0.050 in.

The portion of the actuation member 550 comprising the electrically insulative material (i.e., the electrically insulative portion 560) is made of a material that is electrically insulative, while being strong enough to transmit the actuating forces (i.e. tensile and/or compressive forces) while being sufficiently sized to fit within joint tube portion 545. For example, an amount of space or tolerance available to couple electrically insulative portion 560 with the actuation member 550 gets smaller as the electrically insulative portion is disposed within the distal portions of shaft 532 versus the more proximal portions of shaft 532 as described in, for example, FIG. 3. For example, whereas the embodiment of FIG. 3 described the electrically insulative portion including overmolds on its either side, the tolerances within joint tube portion 545 may not allow for such overmolds. Thus, the electrically insulative portion 560 may be coupled to actuation member 550 using, for example, crimped hypotubes as further described in FIGS. 6A-6B. Further, whereas a proximally-disposed electrically insulative portion (described in, for example, FIGS. 3-4) utilizes materials such as Amodel for its construction, the electrically insulative portion 560 provided in the joint tube portion 545 may be made of a combination of a fiberglass and plastic material that is able to be crimped with said crimped hypotubes. Such a non-conductive plastic/fiberglass hybrid material may be able to withstand the required mechanical loads caused by the actuating forces. Other non-conductive materials used in electrically insulative portion 960 may include fiberglass pultrusions (e.g., S-Glass fiberglass), high performance polyaryletherketones such as PEEK, lay-up composite polymers such as KyronMAX, epoxy-fiberglass combinations, and ceramics such as Alumina.

Thus, a rigid electrically insulative portion 560 disposed in between flexible electrically conductive portions 556, 558 is able to form an electrical break in a conductive pathway between electrically conductive portions 556 and 558. Consequently, a length of a conductive pathway between end effector 540 and a proximal end of surgical instrument 530 is reduced by provision of electrically insulative portion 560. As an amount of capacitive coupling induced in an actuation member (by, for example, nearby surgical instruments that are energized with electrical energy) is proportional to a conductive length of the actuation member, the shorter conductive pathway of the disclosed embodiment (i.e. between end effector 540 and electrically insulative portion 560) minimizes a capacitive coupling effect in a direction proximal from insulative portion 560.

FIGS. 6A and 6B show schematic views of an exemplary embodiment of an actuation member 650 with an electrically insulative portion 660 disposed therein. For example, electrically insulative portion 660 is configured to provide electrical isolation between a distal portion and a proximal portion of the actuation member 650. In this exemplary embodiment, actuation member 650 comprises a first portion 656 distal to electrically insulative portion 660 and a second portion 658 proximal to electrically insulative portion 660. Further, electrically insulative portion 660 is coupled to each of first portion 656 and second portion 658 using crimped hypotubes 663, 664 respectively. For example, a hypotube 663 is provided over the first portion 656 of actuation member 650 and a distal end of electrically insulative portion 660, and crimped so as to create a coupling. Similarly, a hypotube 664 is crimped over the second portion 658 of actuation member 650 and a proximal end of electrically insulative portion 660. Further, first portion 656 and second portion 658 of the actuation member 650 may comprise a flexible electrically conductive material enabling translation and/or articulation of a component of a surgical instrument, such as an end effector or a joint structure (not shown herein). For example, a shaft (not shown herein) housing electrically conductive portions 656, 658 may include one or more joint structures that impart one or more degrees of freedom to an end effector. Consequently, portions 656, 658 of the actuation member 650 may be made of a flexible material such as, for example, a cable having a degree of flexibility sufficient to enable flexion with the translation and/or articulation of an end effector or joint structure.

Further, electrically insulative portion 660 is made of a rigid non-conductive material, so as to enable transmission of force between flexible portions 656, 658. As further described herein, despite flexible portions 656, 658 of the actuation member being routed through the aforementioned joint structures, electrically insulative portion 660 may be disposed within a portion of the length of the actuation member that remains straight during a range of motion (or “throw”) of the actuation member 650. For example, the electrically insulative portion 660 may be disposed in a region corresponding to a joint tube portion (such as, for example, joint tube portion 545 in FIG. 5). Further, electrically insulative portion 660 is formed from a nonconductive material that is sufficiently strong to transmit an actuating force (including push and pull forces) that are delivered from a drive mechanism.

Thus, a rigid electrically insulative portion 660 disposed in between flexible electrically conductive portions 656, 658 is able to form an electrical break in a conductive pathway between electrically conductive portions 656 and 658. Consequently, a length of a conductive pathway between a distal end and a proximal end of actuation member 650 is reduced by provision of electrically insulative portion 660. As an amount of capacitive coupling induced in an actuation member (by, for example, nearby surgical instruments that are energized with electrical energy) is proportional to a conductive length of the actuation member, the shorter conductive pathway of the disclosed embodiment minimizes a capacitive coupling effect in a direction proximal from insulative portion 660. Further, an electrically insulative sleeve 661 is disposed over the electrically insulative portion 660 and extends proximally and distally beyond crimped hypotubes 663, 664, thereby forming a continuous electrically insulated outer surface. The electrically insulative sleeve 661 comprises a sleeve (e.g., tube) of material configured to tightly contract around electrically insulative portion 660 and crimped hypotubes 663. For example, the electrically insulative material may be heat-shrink tubing made of, for example, nylon, polyolefin, or other heat-shrinkable and electrically insulative polymer materials.

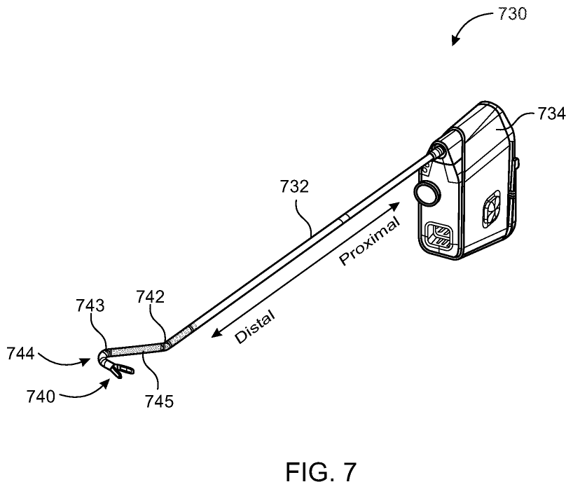

As described above, a shaft of an instrument may optionally include one or more joint structures that impart one or more degrees of freedom to an end effector coupled to a distal end of the instrument. FIG. 7 shows an exemplary embodiment of an instrument 730 including one or more joint structures provided in a shaft 732. For example, as shown in FIG. 7, the one or more joint structures include a pitch/yaw joints 742, 743 (with the terms “pitch” and “yaw” being arbitrarily defined), and a jointed wrist portion 744. For example, a pitch joint is configured to translate the end effector 740 in a first plane of rotation, a yaw joint is configured to translate the end effector 740 in a second plane of rotation, and wrist portion 744 is configured to articulate end effector 740 in various directions. Further, the portion of shaft 732 located in between pitch/yaw joints 742 and 743 may be referred to as a joint tube portion 745.

Thus, in various exemplary embodiments, a proximal portion of an actuation member extending through a non-jointed portion of shaft 732 may be relatively rigid to be able to interface with and transmit force from force transmission mechanism 734, while a distal portion of the actuation member extending through jointed structures 742, 743, 744 may comprise a flexible portion. The flexible portion of the actuation member may comprise a cable (e.g., such as twisted or braided strands of metal or metal alloy), having a degree of flexibility sufficient to enable the flexible portion to flex (e.g., bend) with the translation and/or articulation of joint structures 741. Further, the actuation member may comprise an electrically insulative portion made of a rigid non-conductive material, so as to enable transmission of force through joint tube portion 745. For example, as described above with reference, for example, to FIG. 5), despite flexible portions of the actuation member being routed through the aforementioned joint structures, the electrically insulative portion may be disposed within a portion of the length of the flexible actuation member that remains straight during a range of motion (or “throw”) of the actuation member, i.e. within a region corresponding to joint tube portion 745. Thus, the electrically insulative portion is formed from a nonconductive material that is sufficiently strong to transmit an actuating force (including push and pull forces) that are delivered from drive mechanism 734.

As described above, an electrically insulative “break” may be provided in components of an instrument other than an actuation member. For example, the capacitive coupling effect described above is induced in various conductive components of an instrument due to electrical energy in the actuation member. These various components include metal tubes, such as the main shaft of the instrument, the distal main tube, parallel motion mechanism tube, and other generally elongate components of the surgical instrument. Similar to the actuation member, the capacitance of these electrically conductive components is directly proportional to their length. Thus, additional exemplary embodiments include electrically conductive (e.g., metal) components comprising insulative breaks to reduce a conductive length of the component, thereby reducing the capacitance thereof.

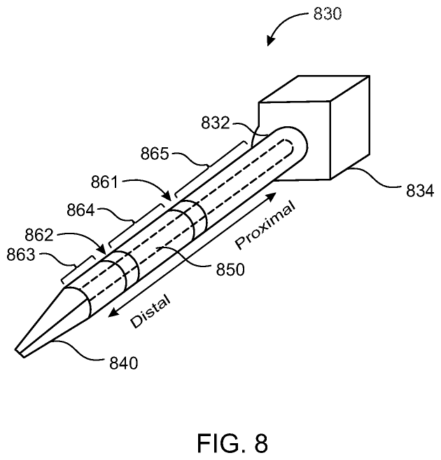

FIG. 8 is a perspective, diagrammatic view of an exemplary embodiment of a surgical instrument comprising a shaft having an electrically insulative portion in accordance with the present disclosure. The surgical instrument 830 comprises a shaft 832 with an end effector 840 coupled at a distal end region thereof. A force transmission mechanism 834 coupled at a proximal end region of the shaft 832 generates actuating forces that are transmitted via one or more actuation members 850 to actuate or articulate various components of the shaft 832 or end effector 840, as discussed above and as those having ordinary skill in the art have familiarity. The diameter or diameters of shaft 832 and end effector 840 are generally selected according to the size of the cannula or other guide structure with which surgical instrument 830 is intended to be used, and depending on the surgical procedures being performed. In various exemplary embodiments, a shaft 832 has a diameter of ranging from about 4 mm to about 10 mm, for example, about 5 mm to about 8 mm. The actuation member 850 may be positioned within a central bore of shaft 832. The actuation member 850 is configured to transmit an actuating force generated at force transmission mechanism 834. For example, the actuation member 850 can be a compression rod-like member or cable member capable of transmitting tensile (i.e. pulling) and/or compressive (i.e. pushing) forces to actuate other components of surgical instrument 830, such as end-effector 840

The shaft 832 comprises an electrically conductive material, (e.g., a metal and/or metal alloy) with one or more electrically insulative portions 861, 862 disposed along a portion of the length of the shaft 832. The one or more electrically insulative portions 861 can be dimensioned and arranged so as to form an electrically insulative “break” in a conductive pathway along a length of the shaft 832, such as between the proximal and distal ends of the shaft 832. As a result, the conductive pathway of the shaft 832 may be divided into various sections that are electrically insulated from one another. For example, in the exemplary embodiment illustrated in FIG. 8, the electrically insulative portions 861, 862 are positioned such that shaft 832 comprises three conductive, yet electrically isolated, length portions: 863, 864, and 865. Since the amount of capacitive coupling is further proportional to the length of the instrument shaft itself, reducing a conductive length of the shaft into two or more electrically conductive length portions (that are electrically insulated from each other) can further reduce a capacitive coupling effect induced within the instrument shaft by other energized components of the instrument such as, for example, an energized actuation member. In other exemplary embodiments, one or more electrically insulative portions, such as electrically insulative portions 861 and 862, may be disposed anywhere along a length of the shaft 832 so as to shorten conductive pathways in different areas of the shaft 832. For example, an insulating layer or insulative sheath may be disposed over a first portion of shaft 832, while a second portion of shaft 832 may be exposed. Thus, disposing an electrically insulative portion between the first and second portions electrically insulates the second portion from the first portion, resulting in a reduced capacitive coupling in the second portion of the shaft.

Further, electrically insulative portions 861, 862 are formed from a non-conductive material that is sufficiently strong to provide structural integrity for the shaft. Suitable materials for electrically insulative portions 861, 862 may include, but are not limited to, for example, thermoplastics such as Amodel, high performance polyaryletherketones such as PEEK, lay-up composite polymers such as KyronMAX, epoxy-fiberglass combinations, ceramics such as Alumina, or a polymer based tube.

As described above, there may be additional conductive components of the instrument which cause unintended electrical effects. For example, cable hypotube assemblies that run the length of the instrument may come in contact with the inside walls of the main shaft, which connects parts of the tube intended to be insulated from each other by the electrically insulative portions thereof. Thus, these individual hypotubes may also include electrically insulative portions along their lengths or in specific areas. In other exemplary embodiments, a plurality of conductive components of an instrument may be provided with insulative “breaks” that are located to minimize an overall capacitance of the instrument. For example, instruments that have additional cables or wires running down the middle to supply electrical energy (such as bipolar instruments) may cause capacitive coupling to the hypotubes and, thus, the hypotubes (or sections thereof) may be made from electrically insulative materials. For example, a dielectric “break” may be provided in one or more of the hypotube assemblies that span the “break” in the main tube, so that energy capacitively coupled from the center rod or wires to the proximal parts of the main tube and hypotubes is insulated from the distal parts of the hypotubes and main tube. In an exemplary embodiment, an instrument sheath having a diameter of 8 mm may comprise insulative “breaks” made of Vectran.

The above-described exemplary embodiments refer to instruments such as, for example, surgical instruments, but are not limited to such applications. For example, the concept described herein may be applicable to other applications of remotely actuatable instruments in non-surgical settings, where it may be desirable to reduce a capacitive coupling of actuation members inside or outside an instrument, and to generally shorten or control unintended electrical pathways. Further, actuation members according to exemplary embodiments of the disclosure provide electrical insulation between exterior distal and proximal portions of the actuation member, while enabling portions of the actuation member to be constructed from metals or metal alloys with relatively high tensile strength, hardness, and/or toughness. Such construction thereby provides reliable operation and longevity due to the material characteristics of the metals/alloys and (tough insulative materials) between the proximal and distal portions. Such actuation members may also reduce a conductive path between distal and proximal portions of the instrument, thereby reducing or eliminating a capacitive coupling effect that may be induced in said actuation members, in between said the actuation members and other portions of the instrument, such as the instrument shaft, wrist, or other exposed electrically conductive portions.

Generally, the electrically insulative portions of the exemplary actuation members illustrated herein have lengths that are greater than a minimum length based on a dielectric strength of a material used to form the electrically insulative portions. Further, materials selected to form the electrically insulative portions are able to withstand operating temperature of the instrument that may reach 150 degrees Celsius, as well as good arc-tracking properties. In some exemplary embodiments, portions of the surgical instrument that incorporate such an electrically insulative material may be disposable. Such single-use examples may incorporate electrically insulative portions formed from materials that need not be subject to repeated electrical effects, and may be selected based on a strength of the material, or an ability of the material to transmit actuating forces to an end effector. Examples of such materials include glass-filled polymers, ceramics, etc. Further, in exemplary embodiments where the electrically insulative portions are provided closer to a distal end of the surgical instrument, tolerances are smaller (e.g., approximately 0.01 in.-0.3 in. in diameter and 0.8 in.-1 in. long), and different materials may be used that may be stronger than injection-molded plastic, such as fiberglass manufactured using pultrusion (e.g., S-Glass fiberglass) and encapsulated within an epoxy resin. With the smaller tolerances, crimping may be utilized to couple electrically insulative portions with electrically conductive portions. Thus, materials that can withstand crimping may be used in these exemplary embodiments, including machined sapphire, blow-coated ceramic, and combinations thereof, such as a metal coated with a thin layer of ceramic, with the thickness of the ceramic layer being thin enough to withstand crimping.

This description and the accompanying drawings that illustrate exemplary embodiments should not be taken as limiting. Various mechanical, compositional, structural, electrical, and operational changes may be made without departing from the scope of this description and the invention as claimed, including equivalents. In some instances, well-known structures and techniques have not been shown or described in detail so as not to obscure the disclosure. Like numbers in two or more figures represent the same or similar elements. Furthermore, elements and their associated features that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

1.-39. (canceled)

40. An actuation member for transmitting a force from a drive mechanism along a shaft of an instrument to an end effector, the actuation member comprising:
a longitudinal axis defining an axial direction of the actuation member;
a first electrically conductive length portion;
a second electrically conductive length portion positioned proximally relative to the first electrically conductive length portion; and
an electrically insulative length portion disposed between and connecting the first electrically conductive length portion and the second electrically conductive length portion,
wherein the actuation member is configured to transmit force along a proximal-distal direction of each of the first electrically conductive length portion, the second electrically conductive length portion, and the electrically insulative length portion from the drive mechanism to the end effector, and
wherein the second electrically conductive length portion has a longer length than the first electrically conductive length portion.

41. The actuation member of claim 40, wherein the electrically insulative length portion is disposed between a proximal end of the first electrically conductive length portion and a distal end of the second electrically conductive length portion.

42. The actuation member of claim 41, wherein the electrically insulative length portion is respectively coupled to the proximal end of the first electrically conductive length portion and the distal end of the second electrically conductive length portion by a crimp.

43. The actuation member of claim 40, wherein one or both of the first and second electrically conductive portions comprise flexible cables.

44. The actuation member of claim 40, wherein the electrically insulative portion comprises fiberglass.

45. The actuation member of claim 44, wherein the fiberglass is pultruded.

46. The actuation member of claim 44, wherein the electrically insulative length portion comprises an epoxy layer covering the fiberglass.

47. An actuation member for transmitting a force from a drive mechanism along a shaft of an instrument to an end effector, the actuation member comprising:
a longitudinal axis defining an axial direction of the actuation member;
a first electrically conductive length portion;
a second electrically conductive length portion positioned proximally relative to the first electrically conductive length portion; and
an electrically insulative length portion disposed between and connecting the first electrically conductive length portion and the second electrically conductive length portion,
wherein the actuation member is configured to transmit force along a proximal-distal direction of each of the first electrically conductive length portion, the second electrically conductive length portion, and the electrically insulative length portion from the drive mechanism to the end effector, and
wherein the electrically insulative length portion is coupled to the first and second electrically conductive length portions using crimped hypotubes.

48. The actuation member of claim 47, wherein the electrically insulative length portion is disposed between a proximal end of the first electrically conductive length portion and a distal end of the second electrically conductive length portion.

49. The actuation member of claim 48, wherein the electrically insulative length portion is respectively coupled to the proximal end of the first electrically conductive length portion and the distal end of the second electrically conductive length portion using the crimped hypotubes.

50. The actuation member of claim 47, wherein the electrically insulative portion comprises fiberglass.

51. The actuation member of claim 50, wherein the fiberglass is pultruded.

52. The actuation member of claim 47, wherein the second electrically conductive length portion has a longer length than the first electrically conductive length portion.

53. An instrument, comprising:
a shaft having a proximal end and a distal end;
an end effector coupled to and extending in a direction distally away from the distal end;
a force transmission mechanism coupled to a proximal region of the shaft; and
an actuation member extending through the shaft and being operably coupled to the force transmission mechanism at one end and to a moveable component of the instrument at an opposite end, wherein the actuation member comprises:
a first electrically conductive length portion;
a second electrically conductive length portion; and
an electrically insulative length portion disposed between and connecting the first electrically conductive length portion and the second electrically conductive length portion.

54. The instrument of claim 53, wherein the shaft further comprises a wrist portion adjacent the distal end, and the actuation member extends through the shaft and the wrist portion.

55. The instrument of claim 54, wherein the wrist portion of the shaft comprises at least two joint portions, and a tube portion provided in between the at least two joint portions.

56. The instrument of claim 55, wherein the electrically insulative length portion is located within the tube portion.

57. The instrument of claim 53, wherein the electrically insulative length portion is disposed in a distal end portion of the shaft proximate the end effector.

58. The instrument of claim 57, wherein the electrically insulative length portion is disposed between 3 inches and 6 inches from the end effector.

59. The instrument of claim 53, wherein the first electrically conductive length portion comprises a rod member, the second electrically conductive length portion comprises a flexible cable, and the electrically insulative length portion is secured to a proximal end of the flexible cable.
